(19) United States
(12) Patent Application Publication
(10) Pub. No.: US 2015/0209337 A1
(43) Pub. Date: Jul. 30, 2015

(54) NICOTINAMIDE DERIVATE IN THE TREATMENT OF ACUTE CORONARY SYNDROME

(71) Applicant: GlaxoSmithKline LLC, Wilmington, DE (US)

(72) Inventors: Michele Ghirardi, Harlow (GB); David Greenhalgh, Harlow (GB); Dennis L. Sprecher, King of Prussia, PA (US); Robert Nicholas Willette, King of Prussia, PA (US)

(21) Appl. No.: 14/414,798

(22) PCT Filed: Jul. 9, 2013
(86) PCT No.: PCT/US13/49703

§ 371 Date: Jan. 14, 2015

Related U.S. Application Data
(60) Provisional application No. 61/672,439, filed on Jul. 17, 2012.

(51) Int. Cl. A61K 31/44; A61K 31/724; A61K 45/06

(52) U.S. Cl. CPC A61K 31/44; A61K 45/06; A61K 31/724

(57) ABSTRACT

The present invention relates to the use of a nicotinamide derivative in the treatment acute coronary syndrome (ACS) and pharmaceutical compositions used in such treatment.

### FIELD OF THE INVENTION

This invention relates to a new pharmaceutical use of a compound which is known in the art as a p38 kinase inhibitor. More specifically this invention relates to the use of a nicotinamide derivative in the treatment acute coronary syndrome (ACS) and pharmaceutical compositions used in such treatment.

### BACKGROUND OF THE INVENTION

The term acute coronary syndrome (ACS) refers to patients experiencing acute coronary ischemia manifest as unstable angina (UA), ST-segment elevation MI (STEMI), and non-ST segment elevation MI (NSTEMI). ACS is caused by obstructed blood flow in the coronary arteries and is the result of rupture and subsequent thrombosis of atherosclerotic plaques. Each index ACS event is followed by a high rate of major adverse cardiovascular events (MACE) events including recurrent myocardial infarction (MI), stroke, and death. Available therapies have markedly reduced morbidity and mortality over the last 20-30 years; however, the combined rate of death, MI, and stroke remains at least 6% during the 3 months following presentation with ACS, even with optimized therapy in a clinical study.

ACS is recognized to be an inflammatory condition, characterized by elevated levels of C-reactive protein (CRP), a biomarker of systemic inflammation, and by heightened inflammatory activity in atherosclerotic plaques, manifest clinically as plaque rupture in the coronary arteries.

The present standard of care for acute coronary syndrome consists of agents that are used during the acute presentation to the emergency department (e.g. nitates, anti-platelet agents, anti-coagulants and thrombolytics) as well as agents that are prescribed for chronic use after discharge (e.g. beta-blockers, ACE inhibitors). During hospitalization, percutaneous intervention (PCI: stenting and/or angioplasty) and coronary artery bypass grafting (CABG) may also be used for acute care. There remains a need for novel therapies, which fill the gap between the acute and chronic therapies described above, that are targeted to be used in the period (e.g. up to approximately 3 months) immediately following an ACS event.

Patent application WO03/068747 (SmithKline Beecham Corporation) discloses a series of nicotinamide derivatives that are useful as p38 inhibitors. The compound 6-(5-cyclopropylcarbamoyl-3-fluoro-2-methyl-phenyl)-N-(2,2-dimethylpropyl)-nicotinamide is specifically described therein. The statement of non-proprietary name adopted by the USAN Council for this compound is losmapimod.

Cheriyan et al (Circulation. 2011; 123:515-523] discloses that losmapimod improves nitric oxide mediated vasodilatation in hypercholerolesteric patients.

### SUMMARY OF THE INVENTION

In a first aspect there is provided a method of preventing or reducing the risk or severity of a major adverse cardiac event (MACE) in a subject that has previously experienced an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt thereof.

In a second aspect there is provided a method of reducing vascular inflammation and/or stabilising atherosclerotic plaques in a subject that has previously experienced an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt thereof.

In a third aspect there is provided a method for protecting myocardium and improving its function peri and post an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt thereof.

In a fourth aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt thereof for use in the above methods of treatment.

In a fifth aspect there is provided a pharmaceutical composition for use in the above methods of treatment.

### DETAILED DESCRIPTION OF THE INVENTION

In a first aspect there is provided a method of preventing or reducing the risk or severity of a major adverse cardiac event (MACE) in a subject that has previously experienced an acute coronary syndrome (ACS) event comprising administering the compound 6-(5-cyclopropylcarbamoyl-3-fluoro-2-methyl-phenyl)-N-(2,2-dimethylpropyl)-nicotinamide that is to say, the compound having the formula (I)

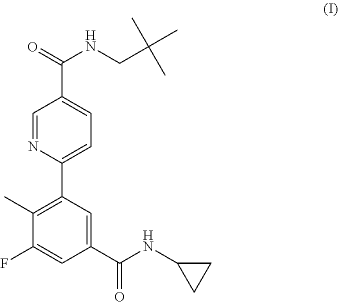 [SMILES: Cc1c(F)cc(C(=O)NC2CC2)cc1-c1ccc(C(=O)NCC(C)(C)C)cn1]

or a pharmaceutically acceptable salt.

In one embodiment there is provided a method of preventing or reducing the risk or severity of a major adverse cardiac event (MACE) in a subject that has previously experienced an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt.

In one embodiment the said MACE is unstable angina (UA). In an alternative embodiment the said MACE is ST segment elevation myocardial infarction (STEMI). In a yet further embodiment the said MACE is non-ST segment elevation myocardial infarction (NSTEMI).

Acute coronary syndrome (ACS events) are typically followed by an acute inflammatory response, which is reflected in significantly elevated levels of inflammatory markers such as C-reactive protein (CRP), cytokine signalling (e.g. IL6) and metalloproteinases (MMP9) and thereby gives rise to a high risk of atherosclerotic plaques rupture; as well as inducing down-stream constriction of the myocardial microvasculature that decreases cardiac perfusion (nutrient supply and oxygen). The latter is specifically supported by Cheriyan et al (Circulation. 2011; 123:515-523],

In a further aspect there is provided a method of reducing vascular inflammation and/or stabilising atherosclerotic plaques in a subject that has previously experienced an acute coronary syndrome (ACS) event comprising administering the compound 6-(5-cyclopropylcarbamoyl-3-fluoro-2-methyl-phenyl)-N-(2,2-dimethylpropyl)-nicotinamide or a pharmaceutically acceptable salt thereof.

In one embodiment there is provided a method of reducing vascular inflammation and/or stabilising atherosclerotic plaques in a subject that has previously experienced an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt thereof.

There is also a need to protect the myocardium and improve its function during the active infarct and the immediate post-infarct healing phase (i.e. peri and post ACS). Myocardium can be protected by permitting improved vascular flow (improving vasoregulation), as well as increasing the threshold for myocardial cell death when under stress (i.e. a reduction in apoptosis), and ultimately by allowing the myocardium to heal post-infarct such that the ventricle maintains its function (i.e. decrease detrimental remodelling).

In a further aspect there is a method for protecting myocardium and improving its function peri and post an acute coronary syndrome (ACS) event comprising administering the compound 6-(5-cyclopropylcarbamoyl-3-fluoro-2-methyl-phenyl)-N-(2,2-dimethylpropyl)-nicotinamide or a pharmaceutically acceptable salt thereof.

In one embodiment there is provided a method for protecting myocardium and improving its function peri and post an acute coronary syndrome (ACS) event comprising administering a therapeutically effective amount of the compound 6-(5-cyclopropylcarbamoyl-3-fluoro-2-methyl-phenyl)-N-(2,2-dimethylpropyl)-nicotinamide or a pharmaceutically acceptable salt thereof.

Suitably the subject is a mammal, particularly a human.

As used herein, the term “therapeutically effective amount” means that amount of compound 6-(5-cyclopropylcarbamoyl-3-fluoro-2-methyl-phenyl)-N-(2,2-dimethylpropyl)-nicotinamide or a pharmaceutically acceptable salt thereof that will elicit the biological or medical response that is being sought, for instance, by a researcher or clinician. It will be appreciated that to achieve the required therapeutic effect the optimum dosage will be determined by standard methods taking into account a number of factors such as the age, weight and response of the particular patient, the severity of the condition and the route of administration.

In one embodiment the treatment regime will commence within 1 hour, 12 hours, 24 hours, 48 hours or 96 hours after the ACS event.

In one embodiment the compound 6-(5-cyclopropylcarbamoyl-3-fluoro-2-methyl-phenyl)-N-(2,2-dimethylpropyl)-nicotinamide or a pharmaceutically acceptable salt is administered at regular intervals (e.g. one or more times per day) for a time period of 6 months or less, 3 months or less or 1 month or less from the ACS event. In one embodiment the compound is administered twice per day (bid).

In certain embodiments of the methods provided herein, the compound 6-(5-cyclopropylcarbamoyl-3-fluoro-2-methyl-phenyl)-N-(2,2-dimethylpropyl)-nicotinamide or a pharmaceutically acceptable salt thereof may be administered via different routes and/or in different forms at different times over the course of treatment. For example, in one embodiment the compound 6-(5-cyclopropylcarbamoyl-3-fluoro-2-methyl-phenyl)-N-(2,2-dimethylpropyl)-nicotinamide or a pharmaceutically acceptable salt thereof may be administered via a parenteral route (such as intravenous administration) in the hours and days immediately following the ACS event, followed by administration via a different route (such as oral administration) at later time points. The transfer between such routes of administration may occur as a phased regime or alternatively, be an immediate switch between the two routes of administration. This embodiment allow for rapid administration of the compound in the hours and/or days (e.g. within 12 hours to 3 days) immediately following an ACS event, and thereby providing therapeutic blood levels of the drug to be obtained more rapidly. In addition this also allows for easier administration of the compound to a subject who may be incapacitated or partially incapacitated in the hours and/or days immediately following the ACS event but provides a more suitable regime for the recovery phase in the weeks and months following the ACS event.

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in preventing or reducing the risk or severity of a major adverse cardiac event (MACE) in a subject that has previously experienced an acute coronary syndrome (ACS).

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in reducing vascular inflammation and/or stabilising atherosclerotic plaques.

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in for protecting myocardium and improving its function peri and post an acute coronary syndrome (ACS).

Pharmaceutically acceptable salts of the compound 6-(5-cyclopropylcarbamoyl-3-fluoro-2-methyl-phenyl)-N-(2,2-dimethylpropyl)-nicotinamide are non toxic salts and include examples described in patent application WO03/068747, the contents of which is incorporated by reference. For a review of suitable pharmaceutically acceptable salts see also Berge et al., J. Pharm. Sci., 66:1-19, (1977).

In one embodiment the compound 6-(5-cyclopropylcarbamoyl-3-fluoro-2-methyl-phenyl)-N-(2,2-dimethylpropyl)-nicotinamide is in the form of a free base.

The compound 6-(5-cyclopropylcarbamoyl-3-fluoro-2-methyl-phenyl)-N-(2,2-dimethylpropyl)-nicotinamide or a pharmaceutically acceptable salt thereof may be prepared according to procedures described in patent application WO03/068747 (as example 36), the contents of which are incorporated by reference. Alternatively the compound can be prepared by the methods described herein.

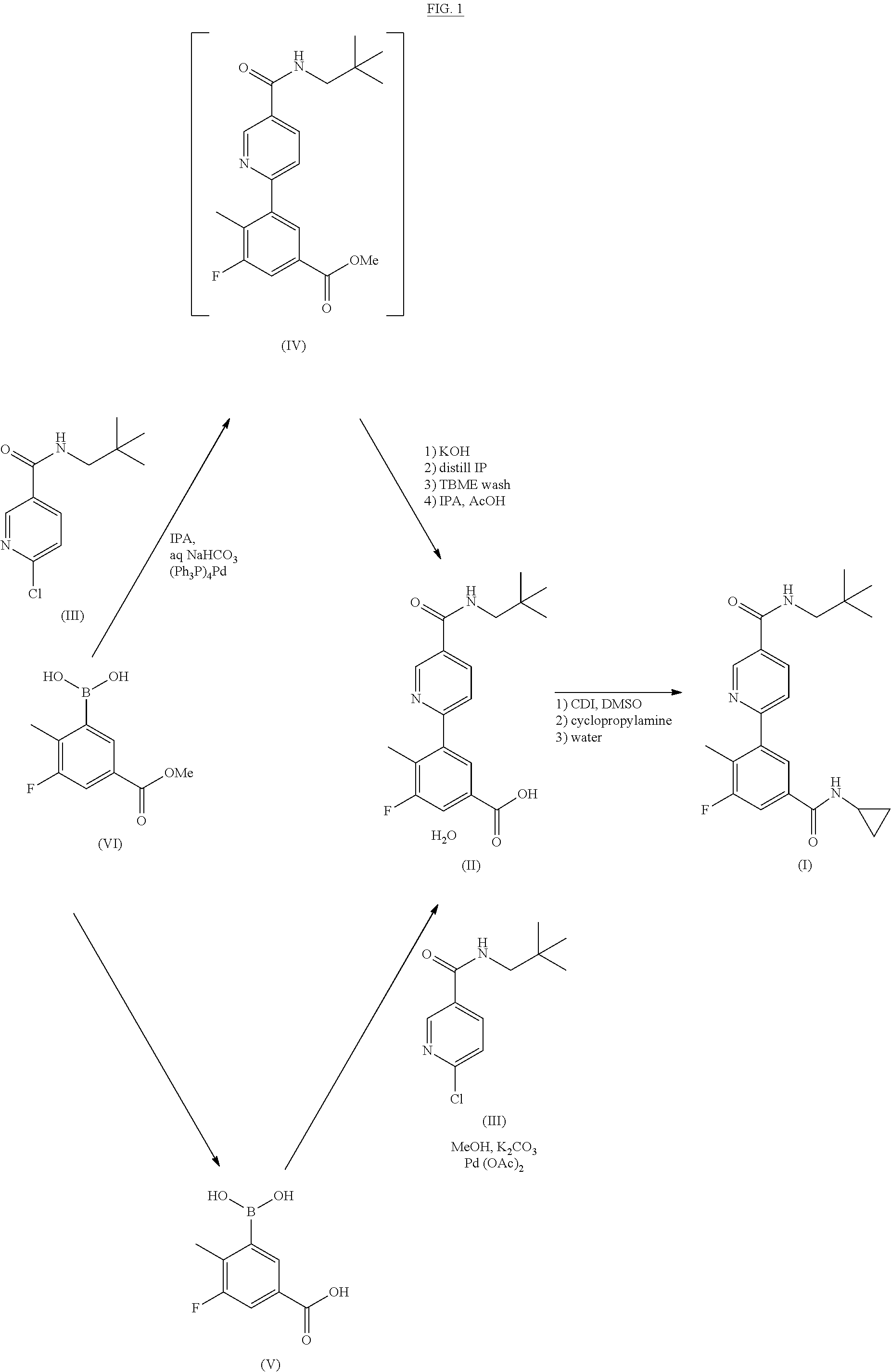

The compound of formula (III) can be prepared by methods described in patent application WO03/068747. The compound of formula (VI) can be prepared by methods described in FIG. 2.

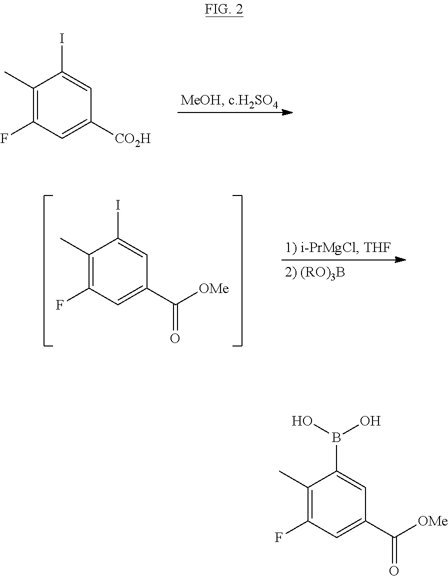

The compound of formula (V) can also be prepared by the methods described in FIG. 3.

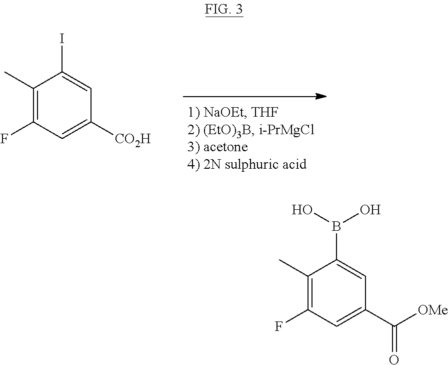

Disclosed is a process for the preparation of a compound of formula (I) which comprises the reaction of a compound of formula (II) with cyclopropylamine amine under amide forming conditions wherein the compound of formula (II) is prepared by reaction of the compound of formula (III) with the compound of formula (V) in the presence of a suitable catalyst (e.g. a palladium catalyst).

Further disclosed is a process for the preparation of a compound of formula (I) which comprises the reaction of a compound of formula (II) with cyclopropylamine amine under amide forming conditions wherein the compound of formula (II) is prepared by reaction of the compound of formula (III) with the compound of formula (VI) in the presence of a suitable catalyst (e.g. a palladium catalyst) and subsequent hydrolysis (e.g. with an aqueous base such as potassium or sodium hydroxide) to the compound of formula (II).

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in preventing or reducing the risk or severity of a major adverse cardiac event (MACE) in a subject that has previously experienced an acute coronary syndrome (ACS).

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in reducing vascular inflammation and/or stabilising atherosclerotic plaques.

In a further aspect of the present invention there is provided the compound 6-(5-cyclopropylcarbamoyl-3-fluoro-2-methyl-phenyl)-N-(2,2-dimethylpropyl)-nicotinamide or a pharmaceutically acceptable salt for use in protecting myocardium and improving its function peri and post an acute coronary syndrome (ACS).

In a further aspect of the present invention there is provided the use of the compound 6-(5-cyclopropylcarbamoyl-3-fluoro-2-methyl-phenyl)-N-(2,2-dimethylpropyl)-nicotinamide or a pharmaceutically acceptable salt in the manufacture of a medicament for use in preventing or reducing the risk or severity of a major adverse cardiac event (MACE) in a subject that has previously experienced an acute coronary syndrome (ACS).

In a further aspect of the present invention there is provided the use of the compound 6-(5-cyclopropylcarbamoyl-3-fluoro-2-methyl-phenyl)-N-(2,2-dimethylpropyl)-nicotinamide or a pharmaceutically acceptable salt in the manufacture of a medicament for use in reducing vascular inflammation and/or stabilising atherosclerotic plaques.

In a further aspect of the present invention there is provided the use of the compound 6-(5-cyclopropylcarbamoyl-3-fluoro-2-methyl-phenyl)-N-(2,2-dimethylpropyl)-nicotinamide or a pharmaceutically acceptable salt in the manufacture of a medicament for use in protecting myocardium and improving its function peri and post an acute coronary syndrome (ACS).

Whilst it is possible for the compound 6-(5-cyclopropylcarbamoyl-3-fluoro-2-methyl-phenyl)-N-(2,2-dimethylpropyl)-nicotinamide or a pharmaceutically acceptable salt thereof to be administered as the raw chemical it would typically be administered in the form of a pharmaceutical composition. 6-(5-cyclopropylcarbamoyl-3-fluoro-2-methyl-phenyl)-N-(2,2-dimethylpropyl)-nicotinamide or a pharmaceutically acceptable salt may therefore be formulated for administration in any suitable manner that is known to those skilled in the art. It may, for example, be formulated for topical administration, transdermal administration, administration by inhalation, oral administration or parenteral administration (e.g. intravenously, intravascularly or subcutaneously). For parenteral administration, the pharmaceutical composition may be given as an injection or a continuous infusion. For administration by injection these may take the form of a unit dose presentation or as a multidose presentation

Suitable methods for formulating 6-(5-cyclopropylcarbamoyl-3-fluoro-2-methyl-phenyl)-N-(2,2-dimethylpropyl)-nicotinamide or a pharmaceutically acceptable salt include those described in patent application WO03/068747 and/or the methods that are familiar to those skilled in the art, which are described in Remington: The Science and Practice of Pharmacy, 21^{st} Edition 2006.

In one embodiment the compound 6-(5-cyclopropylcarbamoyl-3-fluoro-2-methyl-phenyl)-N-(2,2-dimethylpropyl)-nicotinamide or a pharmaceutically acceptable salt thereof is micronised prior to its formulation into a pharmaceutical composition.

In one embodiment 6-(5-cyclopropylcarbamoyl-3-fluoro-2-methyl-phenyl)-N-(2,2-dimethylpropyl)-nicotinamide or a pharmaceutically acceptable salt thereof is adapted for oral administration.

In one embodiment there is provided a pharmaceutical composition suitable for oral administration comprising 6-(5-cyclopropylcarbamoyl-3-fluoro-2-methyl-phenyl)-N-(2,2-dimethylpropyl)-nicotinamide or a pharmaceutically acceptable salt thereof in the form of a tablet having a core which comprises one or more of suitable excipients selected from the group consisting of diluents (such as lactose monohydrate and/or microcrystalline cellulose), binders (such as povidone), lubricants (such as magnesium stearate), disentegrating agents (such as sodium starch glycolate) and optionally having a film coating (such as an Opadry coating).

In one embodiment the tablet core comprises an intra-granular fraction intermingled with an extra-granular fraction. In a particular embodiment the extra-granular component comprises a lubricant.

In one embodiment 6-(5-cyclopropylcarbamoyl-3-fluoro-2-methyl-phenyl)-N-(2,2-dimethylpropyl)-nicotinamide is present in a concentration of 1-10% w/w of the total formulation typically about 5% w/w.

In one embodiment 6-(5-cyclopropylcarbamoyl-3-fluoro-2-methyl-phenyl)-N-(2,2-dimethylpropyl)-nicotinamide or a pharmaceutically acceptable salt thereof is administered orally with a dosage in the range 2.5 mg twice per day (bid) to 15 mg twice per day (bid), particularly 7.5 mg twice per day (bid).

In a further embodiment 6-(5-cyclopropylcarbamoyl-3-fluoro-2-methyl-phenyl)-N-(2,2-dimethylpropyl)-nicotinamide or a pharmaceutically acceptable salt thereof is adapted for intravenous administration.

The compound 6-(5-cyclopropylcarbamoyl-3-fluoro-2-methyl-phenyl)-N-(2,2-dimethylpropyl)-nicotinamide has been found to have an aqueous solubility of approximately 0.005 mg/ml in the physiological pH range 2-10, which is insufficient solublility to achieve adequate dosing via the intravenous route. Moreover, the solubility profile cannot be sufficiently enhanced by using conventional co-solvents. There is therefore a need for a liquid formulation of said compound that is suitable for this mode of administration which solves this technical problem.

In one aspect there is provided a pharmaceutical composition suitable for intravenous administration comprising 6-(5-cyclopropylcarbamoyl-3-fluoro-2-methyl-phenyl)-N-(2,2-dimethylpropyl)-nicotinamide or a pharmaceutically acceptable salt thereof and one or more cyclodextrin.

In one embodiment the cyclodextrin is a 3-cyclodextrin derivative selected from hydroxyalkyl-β-cylodextrin, and sulfobutylether 3-cylodextrin or mixtures thereof. In a particular embodiment the cyclodextrin is hydroxypropyl-3-cylodextrin.

In one embodiment the concentration of 6-(5-cyclopropylcarbamoyl-3-fluoro-2-methyl-phenyl)-N-(2,2-dimethylpropyl)-nicotinamide in the formulation is about 0.4 mg/ml such as 0.4 mg±0.05 mg/ml.

In a further embodiment the cyclodextrin is present in the composition in an amount from about 5 to 25% w/v or from about 10 to 20% w/v, particularly about 15% w/v.

The composition of the invention may optionally comprise further additives such as a solubilisers, isotonizing agents, buffers etc. In one embodiment the composition further comprises a solubiliser (such as ethanol). In a further embodiment the composition further comprises an isotonizing agent (such as NaCl).

The composition may be prepared according using conventional techniques know to those skilled in the art. It has been found that using a pre-solubilisation step significantly accelerates the formation of the cyclodextrin complex. In a further embodiment there is provided a process for the preparation of a pharmaceutical composition suitable for intravenous administration which comprises:
(a) pre-dissolving the compound 6-(5-cyclopropylcarbamoyl-3-fluoro-2-methyl-phenyl)-N-(2,2-dimethylpropyl)-nicotinamide in a suitable solubliliser (such as ethanol);
(b) contacting the resulting solution with a solution comprising a cyclodextrin and an isotonizing agent to form a cyclodextrin complex.

It will be appreciated that 6-(5-cyclopropylcarbamoyl-3-fluoro-2-methyl-phenyl)-N-(2,2-dimethylpropyl)-nicotinamide may be employed alone or in combination with other therapeutic agents which are suitable for use in the above method of treatment.

In a further aspect there is provided a combination product comprising 6-(5-cyclopropylcarbamoyl-3-fluoro-2-methyl-phenyl)-N-(2,2-dimethylpropyl)-nicotinamide, or a pharmaceutically acceptable salt thereof, together with a further therapeutic agent which is suitable for use in the treatment of acute coronary syndromes. In one embodiment the further therapeutic agent is an Lp-PLA₂ inhibitor such as Darapadib. In a further embodiment the further therapeutic agent is an anti-platelet agent. In a further embodiment the further therapeutic agent is a statin, for example, a statin selected from the group consisting of atorvastatin, fluvastatin, lovastatin, pitavastatin, pravastatin, rosuvastatin and simvastatin.

6-(5-Cyclopropylcarbamoyl-3-fluoro-2-methyl-phenyl)-N-(2,2-dimethylpropyl)-nicotinamide and the other therapeutically active agent(s) may be administered together or separately and, when administered separately, this may occur separately or sequentially in any order. The amounts of 6-(5-cyclopropylcarbamoyl-3-fluoro-2-methyl-phenyl)-N-(2,2-dimethylpropyl)-nicotinamide and the other therapeutically active agent(s) and the relative timings of administration will be selected in order to achieve the desired combined therapeutic effect. In one embodiment the other therapeutically active agent may be administered in accordance with its standard recommended dosage while in another embodiment the other therapeutically active agent may be administered in an amount lower than the recommended dosage.

In a further embodiment there is provided a kit comprising 6-(5-cyclopropylcarbamoyl-3-fluoro-2-methyl-phenyl)-N-(2,2-dimethylpropyl)-nicotinamide, or a pharmaceutically acceptable salt thereof and a further therapeutic agent selected from an anti-platelet agent, an Lp-PLA₂ inhibitor and a statin, In a particular embodiment there is further provided an instructions for use.

The following examples are illustrations of certain embodiments of the invention and cannot be considered as restricting in any way.

### Example 1

### A pharmaceutical formulation of 6-(5-cyclopropylcarbamoyl-3-fluoro-2-methyl-phenyl)-N-(2,2-dimethylpropyl)-nicotinamide suitable for intravenous administration

The composition as described in Table 1 was prepared. 6-(5-Cyclopropylcarbamoyl-3-fluoro-2-methyl-phenyl)-N-(2,2-dimethylpropyl)-nicotinamide was pre-dissolved in ethanol and then diluted with an aqueous/isotoniccyclodextrin solution.

TABLE 1

| | Quantity | | Quantity | |
|---|---|---|---|---|
| Component | (per ml) | | (per 5 ml vial) | |
| 6-(5-cyclopropylcarbamoyl-3-fluoro- | 0.4 | mg | 2.0 | mg |
| 2-methyl-phenyl)-N-(2,2- | | | | |
| dimethyl propyl)-nicotinamide | | | | |
| Ethanol | 0.05 | ml | 02.5 | ml |
| Hydroxypropyl Betadex (Kleptose - | 150 | mg | 750 | mg |
| HydroxyPropyl Beta-Cyclodextrin) | | | | |
| NaCl | 5.0 | mg | 25 | mg |
| Water for injection | To 1.0 | ml | To 5 | ml |

The prepared formulation showed good physical and chemical stability at a concentration of active agent which is around 100 fold more concentrated than its aqueous solubility.

### Example 2

### A pharmaceutical formulation of 6-(5-cyclopropylcarbamoyl-3-fluoro-2-methyl-phenyl)-N-(2,2-dimethylpropyl)-nicotinamide suitable for oral administration

The composition as described in Table 2 was prepared.

TABLE 2

| | | % |
|---|---|---|
| Component | mg/tablet | w/w |
| Intragranular | | |
| 6-(5-cyclopropylcarbamoyl-3-fluoro-2-methyl- | 7.5 | 5.0 |
| phenyl)-N-(2,2-dimethylpropyl)-nicotinamide | | |
| (micronized) | | |
| Lactose Monohydrate | 67.9 | 45.3 |
| Microcrystalline Cellulose | 30.0 | 20.0 |
| Sodium Starch Glycolate | 4.5 | 3.0 |
| Povidone | 4.5 | 3.0 |
| Extragranular | | |
| Microcrystalline Cellulose | 30.0 | 20.0 |
| Sodium Starch Glycolate | 4.5 | 3.0 |
| Magnesium Stearate | 1.125 | 0.75 |
| Core Compression Weight | 150 mg | |
| Film Coat | | |
| Opadry White OY-S-28876 | 4.5 | 3.0 |

### Example 3

### In-Vivo Macrophage Activity Study

Macrophage activity and presence are critical features to the vulnerability of plaque in the vasculature. The pivotal nature of p38 MAPK in signaling stress, and its presence in macrophages can be monitored by labeling glucose (fluorodeoxyglucose), an otherwise key nutrient for macrophage activity, and observing its uptake in macrophages using CT imaging techniques. A double-blind, placebo-controlled, parallel group study to evaluate the effects of two regimens of 6-(5-cyclopropylcarbamoyl-3-fluoro-2-methyl-phenyl)-N-(2,2-dimethylpropyl)-nicotinamide (Losmapimod) over a period of 3 months, on in-vivo macrophage activity, as assessed by FDG-PET/CT imaging, in the carotid arteries and aorta of subjects with established atherosclerosis.

### Objectives:

The primary objective was to measure in-vivo macrophage activity, by fluorodeoxyglucose (FDG) positron emission tomography (PET)/computed tomography (CT) imaging, in carotid arteries and aorta following a 12-week treatment with losmapimod (7.5 mg once daily [QD] and 7.5 mg twice daily [BID]), in the setting of chronic statin therapy, as compared to placebo. Secondary objectives included safety and tolerability of 12 weeks of dosing with losmapimod (7.5 mg QD or 7.5 mg BID). Inflammatory biomarkers and the effect of losmapimod 7.5 mg QD vs 7.5 mg BID on in-vivo macrophage activity, as assessed by FDG-PET/CT imaging.

### Population

Ninety-nine patients with vascular inflammation on statins were randomised to losmapimod 7.5 mg once daily (QD), twice daily (BID) or placebo for 84 days. Vascular inflammation was assessed by PET-CT imaging of the carotid arteries and aorta using ¹⁸fluorodeoxyglucose (FDG); the artery with the highest average maximum tissue-to-background ratio (TBR) at baseline (TBR>1.6). 92% of the subjects were white, 86% male, the mean age was 63.8 years (SD 6.13). 72% were current or ex-smokers. All subjects in this study had atherosclerosis as defined in the inclusion criteria; 58% had a history of acute coronary syndrome or myocardial infarction, 24% had a history of transient ischaemic attack or stroke, and 12% have peripheral vascular disease. All subjects were on a stable dose of statin for at least 3 months prior to the first dose and continued on this dose of statin throughout the study.

### Key Findings

The primary end point, change from baseline to day 84 in average maximum TBR was not significantly different between losmapimod and placebo. However exploratory analysis of the imaging data revealed that the proportion of active slices (TBR≧1.6) was significantly reduced from baseline for losmapimod 7.5 mg BID (−9.8%) versus placebo (−6.1%) (p=0.002). Inflammatory biomarkers including high sensitivity C-reactive protein (−28% [95% CI −46, −5]; p=0.023) were significantly reduced for losmapimod 7.5 mg BID versus placebo. FDG uptake was significantly reduced in visceral fat for losmapimod 7.5 mg BID versus placebo (−0.05 [−0.09, −0.01]; p=0.018), but not in subcutaneous fat.

### Conclusion:

Although not meeting the primary efficacy endpoint, Losmapimod decreased vascular inflammation in an atherosclerotic population on statins, concurrent with a reduction in inflammatory biomarkers and FDG uptake in visceral fat. These multiple features suggest a systemic effect which would benefit an ACS setting.

### Example 4

### Study to demonstrate the safety and effects on 6-(5-cyclopropylcarbamoyl-3-fluoro-2-methyl-phenyl)-N-(2,2-dimethylpropyl)-nicotinamide (losmapimod) on inflammatory markers, infarct size and cardiac function on subjects with myocardial infarction

In a randomised, double-blind, placebo controlled study, patients (n=approximately 500) who were admitted to a hospital with the diagnosis of acute coronary syndrome (specifically with non-ST elevation EKG findings upon entry) were provided an oral dose of losmapimod (7.5 mg or 15 mg and thereafter followed by 7.5 mg every 12 hours) or placebo for a period of 3 months.

### Key Findings

a. During the average 4.5 day stay initially in the hospital, particularly in the setting of stent placement the level of inflammation (i.e. CRP and IL6 levels) was determined to be >50% lower with treatment compared to placebo. It is believed that this reduction to be based on limiting the inflammation during both the ongoing infarct and the damage induced by the stent.
b. Secondly, the number of recurrent myocardial infarctions (or episodes of ACS) within the first few months beyond the index infarct, whilst not being statistically significant, trended lower by over 15% with treatment compared to placebo. These data are consistent with the stabilisation of vascular plaque.
c. Thirdly, the size of the index infarct (the one which brought them to the hospital in the first place), was found to be unchanged between groups according to the temoral release of cardiac muscle enzymes measured every 8 hours. However, in a smaller cohort (n=approximately 90), when a magnetic resonance image 4-5 days after the index infarct was evaluated, the size of the infarct was >20% lower in the treated group versus the placebo group. We note that the infarct initiated and predominantly completed by the time of the enzyme assays, and the image provides a more cumulative read for the full peri-ACS period.
d. Fourth, a surrogate guide for cardiac function (BNP) after 12 weeks of treatment and following the majority of post-ACS cardiac healing reveals a >20% reduction in its levels i.e. implying improved cardiac health and function. The three month MR imaging remains supportive of this conclusion given improved function and smaller overall cardiac dimensions. It might be anticipated that fewer heart failure events would be observed.

### Conclusion

These data generally support aspects of the invention suggesting that 6-(5-cyclopropylcarbamoyl-3-fluoro-2-methyl-phenyl)-N-(2,2-dimethylpropyl)-nicotinamide (losmapimod) has a protective effect during the ACS episode and immediately beyond.

All publications, including but not limited to patents and patent applications, cited in this specification are herein incorporated by reference as if each individual publication were specifically and individually indicated to be incorporated by reference herein as though fully set forth.

1. A method of preventing or reducing the risk or severity of a major adverse cardiac event (MACE) in a subject that has previously experienced an acute coronary syndrome (ACS) event comprising administering the compound 6-(5-cyclopropylcarbamoyl-3-fluoro-2-methyl-phenyl)-N-(2,2-dimethylpropyl)-nicotinamide or a pharmaceutically acceptable salt thereof.

2. A method according to claim 1 in which said MACE is unstable angina (UA).

3. A method according to claim 1 in which said MACE is ST segment elevation myocardial infarction (STEMI).

4. A method according to claim 1 in which said MACE is non-ST segment elevation myocardial infarction (NSTEMI).

5. A method of reducing vascular inflammation and/or stabilising atherosclerotic plaques in a subject that has previously experienced an acute coronary syndrome (ACS) event comprising administering the compound 6-(5-cyclopropylcarbamoyl-3-fluoro-2-methyl-phenyl)-N-(2,2-dimethylpropyl)-nicotinamide or a pharmaceutically acceptable salt thereof.

6. A method for protecting myocardium and improving its function peri and post an acute coronary syndrome (ACS) event comprising administering the compound 6-(5-cyclopropylcarbamoyl-3-fluoro-2-methyl-phenyl)-N-(2,2-dimethylpropyl)-nicotinamide or a pharmaceutically acceptable salt thereof.

7. A method according to claim 1 in which the compound is in the form of a free base.

8. A method according to claim 1 in which the compound is administered intravenously.

9. A method according to claim 1 in which the compound is administered orally.

10. A method according to claim 9 in which the compound is administered for a period of 3 months after said acute coronary syndrome (ACS) event.

11. A method according to claim 1 in which the compound is administered in combination with a further therapeutic agent.

12. A method according to claim 11 in which the compound is administered in combination with an anti-platelet agent.

13. A pharmaceutical composition suitable for intravenous administration comprising 6-(5-cyclopropylcarbamoyl-3-fluoro-2-methyl-phenyl)-N-(2,2-dimethylpropyl)-nicotinamide and one or more cyclodextrin.

14. A pharmaceutical composition according to claim 13 in which the cyclodextrin is a β-cyclodextrin derivative selected from hydroxyalkyl-β-cylodextrin, and sulfobutylether β-cylodextrin or mixtures thereof.

15. A pharmaceutical composition according to claim 13 in which the cyclodextrin is hydroxypropyl-β-cylcodextrin.

16. A pharmaceutical composition according to claim 12 in which the concentration of 6-(5-cyclopropylcarbamoyl-3-fluoro-2-methyl-phenyl)-N-(2,2-dimethylpropyl)-nicotinamide in the formulation is about 0.4 mg/ml.

17. A pharmaceutical composition according to claim 12 in which the concentration of cyclodextrin is about 15%.w/v.

18. A process for the preparation of a pharmaceutical composition as defined in claim 12 which comprises:
(a) pre-dissolving the compound 6-(5-cyclopropylcarbamoyl-3-fluoro-2-methyl-phenyl)-N-(2,2-dimethylpropyl)-nicotinamide in a suitable solubliliser;
(b) contacting the resulting solution with a solution comprising a cyclodextrin with an isotonizing agent to form a cyclodextrin complex.
